(19) United States
(12) Patent Application Publication
(10) Pub. No.: US 2010/0248389 A1
(43) Pub. Date: Sep. 30, 2010

(54) ELECTROCHEMICAL DEPOSITION OF POLYMERS ON METAL SUBSTRATES

(75) Inventors: Russell J. Stewart, Salt Lake City, UT (US); Betsy J. Endrizzi, Sandy, UT (US); Gang Huang, Salt Lake City, UT (US); Vladimir Hlady, Salt Lake City, UT (US); Samuel Aaron Poff, Salt Lake City, UT (US); Jennifer Schumaker-Parry, Salt Lake City, UT (US)

Correspondence Address: GARDNER GROFF GREENWALD & VILLANUEVA. PC, 2018 POWERS FERRY ROAD, SUITE 800, ATLANTA, GA, 30339, US

(73) Assignee: UNIVERSITY OF UTAH RESEARCH FOUNDATION, Salt Lake City, UT (US)

(21) Appl. No.: 12/679,050

(22) PCT Filed: Sep. 22, 2008
(86) PCT No.: PCT/US08/77202

§ 371 Date: Jun. 14, 2010

Related U.S. Application Data
(60) Provisional application No. 60/973,826, filed on Sep. 20, 2007.

(51) Int. Cl. G01N 33/53; C25D 11/00

(52) U.S. Cl. 436/501; 205/50; 205/81; 205/317

(57) ABSTRACT

Described herein are methods for electrodepositing a variety of different polymers on metal substrates. The polymers are strongly adhered to the substrates. The substrates produced herein can be used in a number of different applications such as, for example, medical devices and biosensors. For example, the biosensors can be composed of one or more electrodes, where the electrodes have the same or different polymers electrochemically deposited on them. Finally, the methods described herein permit the evaluation of the electrodeposition process as well as monitor the ability of biomolecules to bind to the electrodeposited polymers.

### CROSS REFERENCE TO RELATED APPLICATION

This application claims priority upon U.S. provisional application Ser. No. 60/973,826, filed Sep. 20, 2007. This application is hereby incorporated by reference in its entirety for all of its teachings.

### ACKNOWLEDGEMENTS

The research leading to this invention was funded in part by the National Institutes of Health, Grant No. GM070826. The U.S. Government has certain rights in this invention.

### BACKGROUND

The electropolymerization of thin polymeric films from solutions of monomers is a convenient means to modify electrode surfaces with both conducting and non-conducting coatings. Interest in this approach has been driven by several factors: availability of a wide range of suitable monomers, polymerization is limited to the surface of the electrode, the film thicknesses can be readily controlled, films are uniform and reproducible, and electrodes of any scale and with complex geometries can be modified. In addition to a wide range of applications in electronic devices, electropolymerized films have been used to add biological functionality to electrode surfaces. For example, redox enzymes have been entrapped near electrode surfaces in amperometric biosensors by electropolymerization of films under mild conditions that preserve enzyme function. Electropolymerized films also have been explored as a means to add biorecognition capability to electrode surfaces for specific immobilization of proteins and for improving the electrical characteristics and the biocompatibility of electrodes implanted for chronic in vivo recording.

In view of the many applications of using substrates with electropolymerized polymers deposited thereon, it would be desirable to have a convenient process for applying polymeric films on metal substrates using preformed polymers instead of electropolymerizing monomers on the surface of the metal substrate. The films should be attached so that even under harsh conditions, the film is not removed from the substrate. Finally, it would be desirable to attach a variety of different polymers to the metal substrate with different chemical and physical properties, including hydrophilicity/hydrophobicity, overall charge, biocompatibility, and the like.

### SUMMARY

Described herein are methods for electrodepositing a variety of different polymers on metal substrates. The polymers are strongly adhered to the substrates. The substrates produced herein can be used in a number of different applications such as, for example, medical devices and biosensors. For example, the biosensors can be composed of one or more electrodes, where the electrodes have the same or different polymers electrochemically deposited on them. Finally, the methods described herein permit the evaluation of the electrodeposition process as well as monitor the ability of biomolecules to bind to the electrodeposited polymers. The advantages of the invention will be set forth in part in the description which follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of this specification, illustrate several aspects described below.

FIG. 1 shows the structures of (a) N-methacryloyl tyrosineamide, (b) acrylamide, and (c) 2-(methacrylamidobutyl)nitrilotriacetic acid (MABNTA) used to produce copolymers useful in the methods described herein.

FIG. 2 shows a schematic of the three-electrode configuration used to monitor copolymer passive adsorption and electrochemical deposition by SPR. The gold SPR sensor surface functioned as the working electrode.

FIG. 3 shows the cyclic voltammetry of tyrosine and poly(acrylamide-co-tyrosineamide) in 0.1 M NaCl (scan rate was 100 mV/s).

FIG. 4 shows the change in refractive index (ΔRIU) of passively adsorbed versus electrodeposited copolymer containing 3 mol % tyrosineamide sidechains. (a) Transfer of SPR sensor into cotyrosineamide polymer solution. (b) Application of 0.6 V potential for 5 min (c) Sensor with passively adsorbed copolymer was washed in 0.1 M NaOH and 1% Triton X for 10 min and reequilibrated in water. (d) Sensor with electrodeposited copolymer was washed with 0.1 M NaOH, and 1% Triton X-100 for 10 min and reequilibrated in water.

FIG. 5 shows the copolymer adsorption versus mol % tyrosineamide sidechains. A) Passive adsorption before (blue bars) and after (purple bars) washing with 0.1M NaOH and 1% Triton X-100. B) Electrodeposition before and after washing. Error bars represent the average +/−SD of at least three experiments.

FIG. 6 shows the non-specific GFP binding to an unmodified (blue) and a sensor modified by electrodeposition of 3 mol % tyrosineamide copolymer (pink). (a) Sensor in 0.05 mg/ml GFP-H₆ solution. (b) Sensor re-equilibrated in PBS (pH 7.4).

FIG. 7 shows (A) Specific binding of GFP-H₆ to modified sensor: (a) sensor placed in 0.05 mg/ml GFP-H₆ without Ni(II); (b) sensor re-equilibrated in PBS; (c) sensor in 0.05 mg/ml GFP-H₆ after metallation with Ni(II); (d) sensor re-equilibrated in PBS; (e) sensor washed with 0.1 M EDTA and re-equilibrated in PBS. (B) Fluorescence of GFP-H₆ bound to gold electrodes. Error bars represent the average +/−SD of at least three experiments.

FIG. 8 shows a schematic of GFP-H₆ protein binding to NTA-Ni(II) complexes incorporated into a poly(acrylamide-co-tyrosineamide-co-MABNTA) film electrodeposited on a gold surface.

FIG. 9 shows a sequential process for electrodepositing different polymers on different electrodes.

FIG. 10 shows arrays with different amounts of electrodes (a) two, (b) four, and (c) eight.

FIG. 11 shows protein binding on passivated and non-passivated electrodes after washing with different fluids.

FIG. 12 shows polymer binding curves in the absence of harsh conditions.

FIG. 13 shows the monitoring of the electrodeposition process in situ.

FIG. 14 shows polymer binding curves with passivated electrodes exposed to harsh conditions.

### DETAILED DESCRIPTION

Before the present compounds, compositions, and/or methods are disclosed and described, it is to be understood that the aspects described below are not limited to specific compounds, synthetic methods, or uses as such may, of course, vary. It is also to be understood that the terminology used herein is for the purpose of describing particular aspects only and is not intended to be limiting.

In this specification and in the claims that follow, reference will be made to a number of terms that shall be defined to have the following meanings:

It must be noted that, as used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

“Optional” or “optionally” means that the subsequently described event or circumstance can or cannot occur, and that the description includes instances where the event or circumstance occurs and instances where it does not. For example, the phrase “optionally substituted lower alkyl” means that the lower alkyl group can or cannot be substituted and that the description includes both unsubstituted lower alkyl and lower alkyl where there is substitution.

References in the specification and concluding claims to parts by weight, of a particular element or component in a composition or article, denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent of a component, unless specifically stated to the contrary, is based on the total weight of the formulation or composition in which the component is included.

Variables such as R¹-R³, X, Ar, and m used throughout the application are the same variables as previously defined unless stated to the contrary.

Described herein are methods for electrochemically depositing polymers on metal substrates. In one aspect, the method comprises electrochemically depositing a polymer on a surface of the metal substrate, wherein the polymer comprises at least one aromatic group, and wherein the aromatic group comprises one hydroxyl group.

One approach to electrochemically depositing the polymers described herein on a metal surface is depicted in FIG. 2. A solution of polymer 100 is prepared and introduced into container 110. The solvent used to prepare the polymer solution can vary depending upon the selection and concentration of polymer. In one aspect, the solvent is water. An article 120 with a metal substrate 130 is introduced into polymer solution 100. Electrodes are next immersed in the polymer solution. Referring to FIG. 2, a counter electrode 150 and reference electrode 160 are immersed in the polymer solution. A working electrode 170 is attached to the surface of the metal substrate 130.

Upon application of current through the polymer solution via the electrodes, the polymer is electrochemically deposited on the surface of the metal substrate. This application is unique when compared to the electropolymerization of monomers on the surface of a metal substrate. In one aspect, the electrochemical deposition step is performed at a potential sufficient to oxidize at least one aromatic group. Not wishing to be bound by theory, it is believed that upon application of current, aromatic oxy radicals are generated, which can form dimers and ultimately high molecular weight insoluble polymers. The polymers can adhere to the metal substrate through a variety of bonding mechanisms depending upon the polymer and metal substrate that are selected. Ultimately, after electrochemical deposition, a polymer film is deposited on the metal substrate, where the polymer is strongly adhered to the metal substrate. Indeed, the polymer-coated substrates produced herein are stable under harsh conditions (e.g., exposure to very basic medium as demonstrated in the Examples). In certain aspects, the number of aromatic hydroxyl groups can determine the amount of polymer that is adsorbed onto the metal substrate. For example, when the aromatic hydroxyl group is derived from a tyrosine residue, the polymer can be composed of less than 5 mol %, or less than 3 mol % tyrosine residues to produce suitable adsorption of the polymer on the metal substrate.

The duration and amount of potential applied through the polymer solution will vary depending upon the polymer selected and the desired amount of polymer to be deposited on the metal substrate. For example, the potential can vary depending upon the selection of the aromatic group and the substituents present on the aromatic group (e.g., electron-withdrawing and -donating groups).

A wide variety of polymers possessing aromatic group(s) bearing hydroxyl groups can be used in the methods described herein. The term “aromatic group” as used herein is any carbon-based aromatic group including, but not limited to, benzene, naphthalene, and other fused or biaryl (e.g., biphenyl) groups. The term “aromatic” also includes “heteroaryl group,” which is defined as an aromatic group that has at least one heteroatom incorporated within the ring of the aromatic group. Examples of heteroatoms include, but are not limited to, nitrogen, oxygen, sulfur, and phosphorus.

Each aromatic group possesses one hydroxyl group. It is also contemplated that the same or different aromatic groups can be incorporated into the polymer. For example, a polymer with phenyl and naphthyl groups can be incorporated into the polymer, wherein the phenyl and/or naphthyl groups comprise one hydroxyl group. In the case when the aromatic group is the same, some of the aromatic groups can have one hydroxyl group. The number of aromatic hydroxyl groups in the polymer also affects the adhesion of the polymer to the metal substrate. In certain aspects, by increasing the number of aromatic hydroxyl groups in the polymer, the adhesion between the polymer and the metal substrate also increases. In one aspect, the aromatic hydroxyl group comprises a phenol group. In another aspect, the phenol group is an ortho- or meta-phenol group. In another aspect, the phenol group is a para-phenol group.

The aromatic groups can be substituted with a number of different groups, which can ultimately affect the potential used to electrochemically deposit the polymer on the metal substrate. For example, the aromatic groups can include one or more different halides, which can reduce the potential needed during electrochemical deposition.

In one aspect, the polymer useful herein has a plurality of aromatic groups bearing hydroxyl groups that are directly attached to a polymer backbone or pendant to the polymer backbone. The term “pendant” is defined herein as the aromatic group attached to the polymer backbone by one or more atoms. The selection of the polymer used to produce the backbone can vary depending upon the desired properties of the coated metal substrate. For example, the polymer can be selected so that it is hydrophobic or hydrophilic. Moreover, homopolymers, copolymers, and block copolymers can be used to achieve the desired properties. In one aspect, the polymer comprises a polyester backbone (e.g., polylactic acid, poly glycolic acid, or PLGA), a polyalkylene backbone (e.g., polyethylene, polypropylene), polyalkylene oxide backbone (e.g., PEG, PEO-PPO, etc.), or a polysaccharide backbone.

In one aspect, polymer is the polymerization product between one or more ethylenically unsaturated monomers. The term “ethylenically unsaturated monomer” is defined herein as a compound having one or more carbon-carbon-double bonds. Examples of ethylenically unsaturated monomers include, but are not limited to, acrylates, methacrylates, vinyl compounds, allyl compounds, and the like. The aromatic group bearing the hydroxyl group can be attached directly to one of the carbon atoms of the carbon-carbon double bond. Upon polymerization, the aromatic group is directly attached to the polymer backbone. Alternatively, the aromatic group bearing the hydroxyl group can be attached to one of the carbon atoms of the carbon-carbon double bond by a linker. In this aspect, the aromatic group is pendant to the polymer backbone upon polymerization of the ethylenic ally unsaturated monomer. Examples of linkers include, but are not limited to, a methylene group [—(CH₂)ₙ—, where n is greater than 1], an ether group (e.g., polyethylene oxide), a polyamine, or other suitable groups. The selection of the linker can vary depending upon the desired properties of the resulting film (e.g., hydrophilicity, polarity, etc.).

In another aspect, the polymer comprises a polyacrylate comprising one or more pendant aromatic groups, wherein at least one of the aromatic groups comprises one hydroxyl group. The polyacrylate is generally produced by the polymerization of monomers including acrylates, methacrylates, acrylamides, and any combination thereof. Thus, the polyacrylate can be a homopolymer or copolymer. In certain aspects, an acrylate, methacrylate, or acrylamide that possesses one or more aromatic groups bearing one hydroxyl group can be polymerized to produce the polymer. In one aspect, the polymer comprises the copolymerization product between (1) a compound comprising the formula II

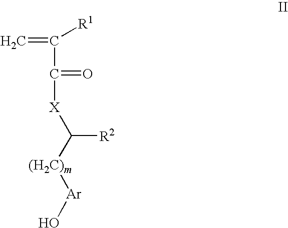

wherein R¹ is hydrogen or an alkyl group;
R² is hydrogen or C(O)NH₂ or C(O)OH;
X is O or NR³, wherein R³ is hydrogen or an alkyl group;
m is from 1 to 10;
Ar is an aromatic group,
or the pharmaceutically-acceptable salt or ester thereof; and
(2) acrylamide, acrylic acid or derivative thereof, methacrylic acid or derivative thereof, and any combination thereof.

In one aspect, wherein R¹ is methyl, X is NH, R² is C(O)NH, m is 1, Ar is a phenyl group, and the OH group is at the para-position of the phenyl group. This monomer is methacrylic tyrosineamide. It is contemplated that other monomers can be used in addition to those recited above. The polymer produced by the polymerization of compound having the formula II comprises at least one fragment comprising the formula I

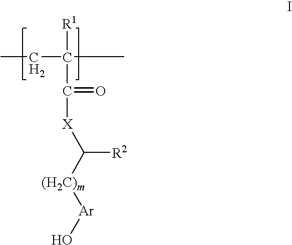

wherein R¹, R², R³, X, m, and Ar are defined above with respect to formula II.

In other aspects, the polymer can be a biomolecule having one or more aromatic groups having at least one hydroxyl group. For example, natural or synthetic proteins and peptides having tyrosine residues or other residues having a phenol group can be electrodeposited on a metal substrate. Other biomolecules include oligonucleotides, small molecules and drugs, and the like.

The polymers can be modified so that the resultant polymer possesses a desired property. For example, different types and amounts of monomers can be polymerized with the monomer having the formula II to modify the properties of the polymer. Examples of these properties include, but are not limited to, hydrophilicity, charge, and binding activity. In one aspect, polymers produced with high amounts acrylamide are relatively hydrophilic. This feature is described in more detail in the Examples.

Any of the polymers described herein can be the pharmaceutically-acceptable salt or ester thereof. In one aspect, pharmaceutically-acceptable salts are prepared by treating the free acid with an appropriate amount of a pharmaceutically-acceptable base. Representative pharmaceutically-acceptable bases are ammonium hydroxide, sodium hydroxide, potassium hydroxide, lithium hydroxide, calcium hydroxide, magnesium hydroxide, ferrous hydroxide, zinc hydroxide, copper hydroxide, aluminum hydroxide, ferric hydroxide, isopropylamine, trimethylamine, diethylamine, triethylamine, tripropylamine, ethanolamine, 2-dimethylaminoethanol, 2-diethylaminoethanol, lysine, arginine, histidine, and the like. In one aspect, the reaction is conducted in water, alone or in combination with an inert, water-miscible organic solvent, at a temperature of from about 0° C. to about 100° C. such as at room temperature. In certain aspects where applicable, the molar ratio of the compounds described herein to base used are chosen to provide the ratio desired for any particular salts. For preparing, for example, the ammonium salts of the free acid starting material, the starting material can be treated with approximately one equivalent of pharmaceutically-acceptable base to yield a neutral salt.

In another aspect, if the polymer possesses a basic group, it can be protonated with an acid such as, for example, HCl, HBr, or H₂SO₄, to produce the cationic salt. In one aspect, the reaction of the polymer with the acid or base is conducted in water, alone or in combination with an inert, water-miscible organic solvent, at a temperature of from about 0° C. to about 100° C. such as at room temperature. In certain aspects where applicable, the molar ratio of the compounds described herein to base used are chosen to provide the ratio desired for any particular salts. For preparing, for example, the ammonium salts of the free acid starting material, the starting material can be treated with approximately one equivalent of pharmaceutically-acceptable base to yield a neutral salt.

Ester derivatives are typically prepared as precursors to the acid form of the compounds. Generally, these derivatives will be lower alkyl esters such as methyl, ethyl, and the like. Amide derivatives —(CO)NH₂, —(CO)NHR and —(CO)NR₂, where R is an alkyl group, can be prepared by reaction of the carboxylic acid-containing compound with ammonia or a substituted amine.

The substrate is generally a metallic material having one or more surfaces for electrochemically depositing the polymer. The selection of the metal substrate can vary depending upon the polymer to be deposited and the potential that is applied. Examples of metal substrates useful herein include, but are not limited to, gold, platinum, palladium, titanium, or iridium. The metals can be elemental metal, an oxide of the metal, or a combination thereof.

The polymer-coated metal substrates produced herein can be used in a number of applications, articles, and devices. In one aspect, the coated metal substrate can be a biosensor. In this aspect, the biosensor comprises a metal substrate having at least one surface and a polymer electrochemically deposited on the surface of the metal substrate, wherein the polymer comprises at least one aromatic group, and the aromatic group comprises one hydroxyl group. In one aspect, the methods described herein can be used to coat metal substrates useful in fluorescent sensors. In another aspect, the coated metal substrates produced herein can be used in a surface plasmon resonance detector or a quartz crystal microbalance.

In other aspects, the biosensor can include one or more electrodes, where each electrode is a polymer-coated metal substrate. In certain aspects, when the biosensor has a plurality of electrodes, each electrode can be composed of the same metal substrate and same polymer. Alternatively, the electrodes can be composed of different metal substrates and/or different polymers.

Depending upon the application of the biosensor, the polymer comprises one or more ligands covalently attached to the polymer. The term “ligand” is defined as any moiety that can form non-covalent bonds with a target (e.g., hydrogen bonding, Lewis acid/base interaction, electrostatic, ionic, and the like). The target can be an ion or a molecule. In one aspect, the ligand comprises a group for chelating metal ions. For example, if the polymer includes one or more amine groups (substituted or unsubstituted), the amine groups can chelate with a metal ion. This aspect is desirable in detecting and measuring the activity of metal ions in biological systems (e.g., enzymatic activity). In one aspect, incorporation of nitrilotriacetic acid (NTA) sidechains into the polymer can specifically immobilize his-tagged proteins. In this aspect, Ni(II) is coordinated to the NTA group, and binding between the [NTA-Ni(II)] and the his-tagged protein can be detected and measured.

In another aspect, the ligand can be a biomarker that specifically binds to a biological molecule. The biomarker can be covalently bonded to the polymer, with the selection of the biomarker depending upon the desired target or reaction to be monitored. Examples of biomarkers include, but are not limited to, a peptide, a protein, an oligonucleotide, or a small molecule (e.g., a pharmaceutical drug). Alternatively, one or more amino acids can be covalently attached to the polymer. In this aspect, the amino acids can be attached to one another to form sidechains or, in the alternative, the amino acids can be attached throughout the polymer. In one aspect, the amino acid comprises tyrosine or tyrosineamide.

Using the techniques described herein, electrodes of any size, shape, and geometric arrangement can be modified in aqueous solution under physiological conditions. Second, because deposition of the polymer is activated by a localized potential, individual electrodes in an electrode array can be selectively modified, which will facilitate the fabrication of ligand arrays. Finally, a wide variety of ligands can be conveniently copolymerized into the polymer, which expands the utility of the biosensor.

In other aspects, the polymer-coated substrates produced herein can be used in a medical device. In one aspect, the medical device comprises a metal substrate having at least one surface and a polymer electrochemically deposited on the surface of the metal substrate, wherein the polymer comprises at least one aromatic group, and the aromatic group comprises at least one hydroxyl group. The polymers deposited on the metal substrate can be selected so that the coated medical device has compatible physiological properties including, but not limited to, hydrophilicity, protein resistance, and biocompatibility. In one aspect, the polymer comprises a polyacrylamide matrix, which creates a hydrophilic surface. In another aspect, one or more biomarkers can be present on the polymer that resists protein binding, which in turn results in reduced biofouling of the medical device.

In other aspects, the methods described herein can produce substrates useful in medical diagnostics and devices. For example, the electrodeposition of hydrophilic polymers on metal substrates can enhance the ability of the substrate to bind to proteins and other biomolecules. As shown in the Examples, the electrodeposited polymers can limit non-specific binding of proteins, which ultimately increases the accuracy, sensitivity, and potential feature density of the device. In certain aspects, the electrodeposition methods described herein may be useful for preparing diagnostic devices and biosensors based on microarrayed ligands. Because adsorption of the polymers to the metal substrate is triggered by a localized potential, it may be possible to selectively modify selected electrodes in an array of electrodes as a means to fabricate ligand microarrays. Polymers copolymerized with unique ligands (e.g., biomarkers) could be sequentially introduced while applying potentials only to the electrode or pattern of electrodes to be sequentially surface modified. Thus, an array of different ligands can be produced.

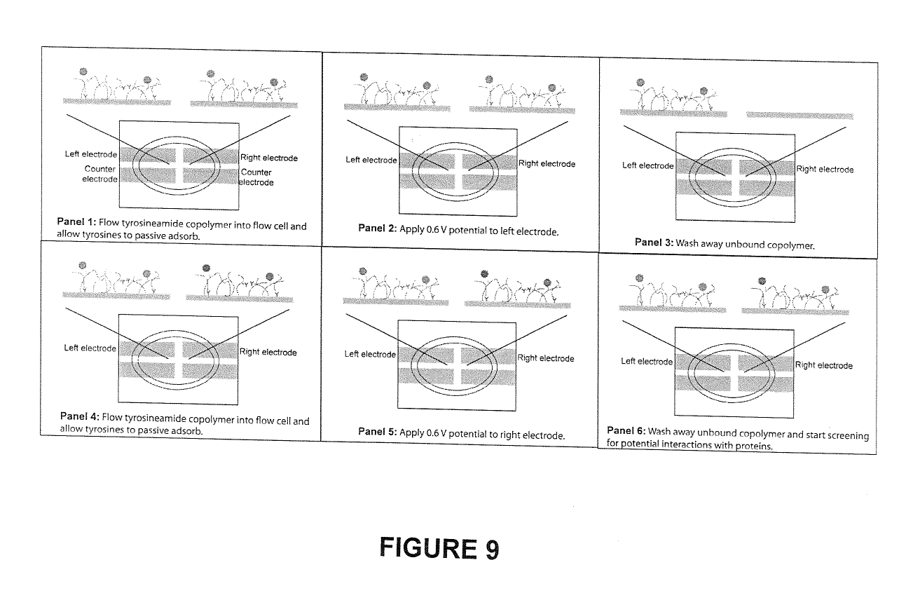

In one aspect, the methods described herein can produce arrays composed of a SPR substrate coated with a plurality of specific ligands, proteins, and the like. By applying an oxidizing potential to the electrode of interest, a particular ligand or protein can be deposited on the electrode that has a specific binding affinity to another molecule (e.g., a second protein). An example of this is depicted in FIG. 9. Panel 1 shows a simplified array with only two elements, the left and right electrodes. A polymer described herein with a specific ligand can be flowed over the surface and passively adsorb to both electrodes. After application of a potential to the left electrode (panel 2) and washing the surface, the right electrode does not have any polymer electrodeposited on the surface while the left electrode does (panel 3). This same process can be repeated using a polymer containing a different type of ligand for the right electrode (panels 4-6). Once the desired positions of the array are filled, a biomolecule of interest such as, for example, DNA or protein, can be placed in the chamber and the interaction between the DNA or protein with a specific ligand on a specific array unit can be monitored and quantified. Thus, in addition to monitoring the electrodeposition of polymers on the surface of arrays, the methods described herein also provide real-time kinetic analysis and equilibrium measurements of the adsorption or binding of biomolecules to the array. As shown in the examples below, impedance measurements can be used to detect and quantify the interaction between the biomolecule of interest and a ligand present on the electrodeposited polymer.

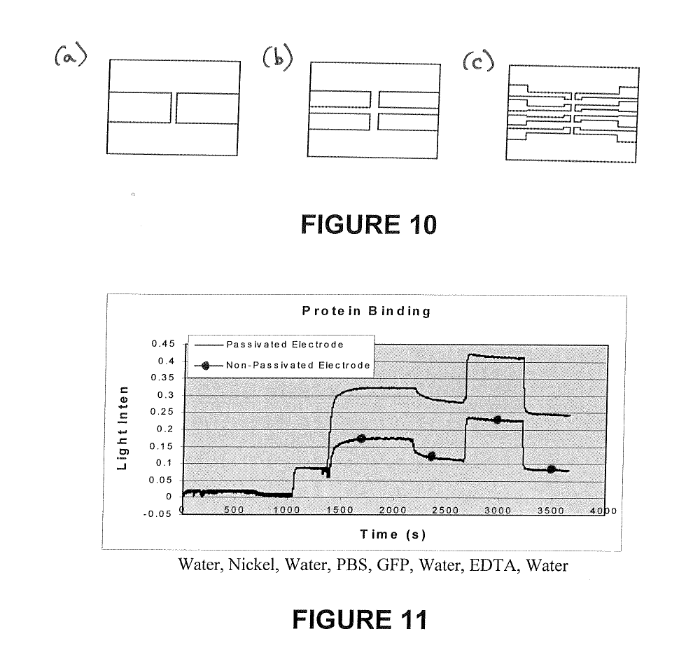

The number and arrangement of electrodes in the array can vary. For example, the number of electrodes can be based upon the detection limit of the instrument used to visualize the electrodes during electrodeposition. Three exemplary array designs are shown in FIG. 10. The first array (a) is a simple design of two electrodes; one as the working electrode and the other as the counter electrode. The second design (b) becomes slightly more complex with four electrodes. The third design (c) is the most complex array with eight electrodes. Optional reference electrodes can be used as well depending upon the application.

As described above, the methods described herein can provide real-time data regarding the electrodeposition of numerous polymers on a metal substrate as well as monitor different types of interactions that can occur on the surface of the substrate. For example, surface plasmon resonance (SPR) is a powerful tool for quantitatively exploring numerous interactions including protein-ligand, protein-protein, protein-DNA, protein-membrane, and more. SPR can achieve high sensitivity without the need for any labeling or fluorescence of the components. As shown in the Examples, SPR can be used to monitor the interaction between gold substrates and various tyrosineamide copolymers. In one aspect, surface plasmon resonance microscopy (SPRM) or surface plasmon resonance imaging (SPRi) can be used as a detection method for multiplex imaging of an electrode array produced by the methods described herein. In other aspects, SPRM can be used to monitor electropolymerization at each electrode in the array individually and simultaneously, in real time in situ, which can be important in the design of arrays and sensors having a plurality of different polymers electrodeposited on the metal surface.

It is understood that any given particular aspect of the disclosed compositions and methods can be easily compared to the specific examples and embodiments disclosed herein, including the non-polysaccharide based reagents discussed in the Examples. By performing such a comparison, the relative efficacy of each particular embodiment can be easily determined. Particularly preferred compositions and methods are disclosed in the Examples herein, and it is understood that these compositions and methods, while not necessarily limiting, can be performed with any of the compositions and methods disclosed herein.

### EXAMPLES

The following examples are put forth so as to provide those of ordinary skill in the art with a complete disclosure and description of how the compounds, compositions, and methods described and claimed herein are made and evaluated, and are intended to be purely exemplary and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### I. Experimental

Materials. Tyrosineamide and Triton X-100 were purchased from Sigma-Aldrich (St. Louis, Mo.). The remaining chemicals and solvents were purchased from Acros (Geel, Belgium) and used as received unless otherwise noted.

Monomer synthesis. The methacrylated NTA group, 2-(methacrylamidobutyl)nitrilotriacetic acid (MABNTA), was synthesized according to Tang, A.; Wang, C.; Stewart, R. J.; Kopecek, J. J. Controlled Release 2001, 72, 57-70. The tyrosineamide monomer (N-methacryloyl tyrosineamide) was synthesized by dissolving N-hydroxysuccinimide (2.31 g, 20 mmol) in 60 ml THF and the pH was adjusted to ˜8 with N—N′-diisopropylethylamine (DIPEA). This solution was cooled to between −5 and 0° C. and, while stirring, distilled methacryloyl chloride (1.9 ml, 19 mmol) was added dropwise. After addition, the mixture was stirred at room temperature for 1 hr. The reaction was again cooled on ice and tyrosineamide (3.6 g, 20 mmol) in DMF was added dropwise. The reaction was stirred overnight at room temperature. The solvents were removed by rotary evaporation. Approximately 80 ml of water was added to the powder and extracted with 4×80 ml ethyl acetate. The ethyl acetate fractions were combined and the solvent was removed by rotary evaporation. Fifty ml of water was added to the residue, which was filtered and washed with approximately 500 ml of water. The filtrate was lyophilized and analyzed by HPLC.

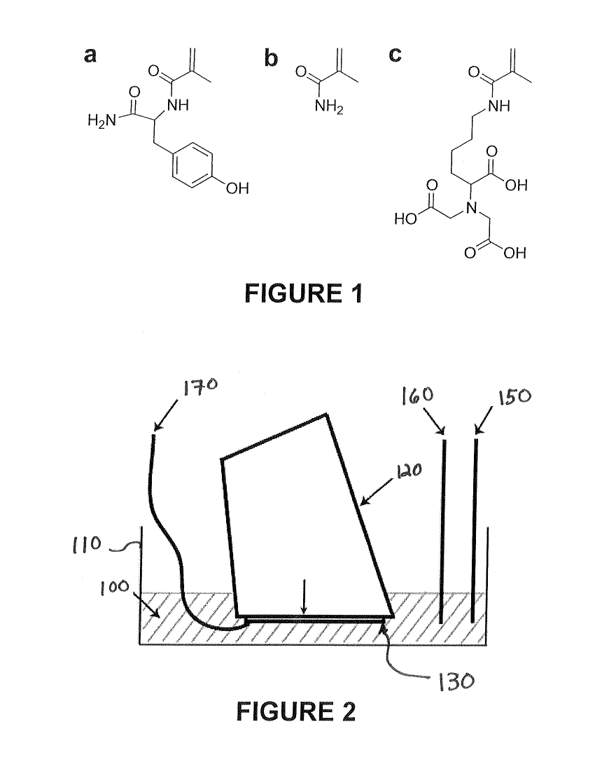

Copolymer synthesis. Poly(acrylamide-co-tyrosineamide) was synthesized by free radical copolymerization of acrylamide with 0 to 6 mol % N-methacryloyl tyrosineamide at 50° C. in methanol (90 wt %) with 2,2′-azobisisobutyronitrile (AIBN, 0.5 wt %) as the initiator under nitrogen for 24 hours. The copolymer was dialysed against water for two days before lyophilization. The poly(acrylamide-co-tyrosineamide-co-MABNTA) copolymers were synthesized by the same procedure using 3% tyrosineamide and 3% MABTA. The structures of the monomers are shown in FIG. 1.

Cyclic voltammetry and impedance measurements. A three-electrode cell with a 0.3 mm Pt wire counter electrode and a 0.2 mm Ag/AgCl reference electrode was used for all electrochemical experiments. Planar Au electrodes (1×1 cm square) were created by sputtering Au on mylar using a TMV Super Series SS-40C-1V Multi-Cathode sputtering system. Ti, used as the seed layer, was deposited at 90 W for 5 min followed by deposition of Au at 90 W for 4 min at 20 mTorr in Ar gas. A Pine Instrument Company (Grove City, PN) RDE4 potentiostat driven by a BNC-2090 terminal block from National Instruments (Austin, Tex.) was used in CV experiments. The planar Au electrodes were placed in the background solution of 0.1 M NaCl and the current was scanned from −0.9 V to +0.9 V at 100 mV/s. The electrodes were placed in a 0.2 wt % polymer solution of the 6 mol % tyrosineamide copolymer in 0.1 M NaCl and the current was measured from −0.9 V to +0.9 V at 100 mV/s. Contact angles were measured by pipetting 20 μL of 18 M-Ohm water onto the same spot of the electrode before and after electrodeposition of the copolymer.

Disk electrodes used for impedance measurements were prepared similar to that disclosed in Zhang, B.; Zhang, Y.; White, H. S. Anal. Chem. 2004, 76, 6229-6238. A 1 cm piece of 0.1 mm 99.99% Au wire (Simga-Aldrich, St. Louis, Mo.) was electrically contacted to a W rod using Ag conductive adhesion paste (Alfa Aesar, Ward Hill, Mass.). This was placed in an oven at 150° C. for 5 min to dry the Ag paste. The Au wire was inserted into a 10 cm length Prism glass capillary (1.65 mm outer diameter, 0.75 mm inner diameter, softening point 700° C., Dagan Corporation, Minneapolis, Minn.) leaving ˜2 mm space between the tip of the Au wire and the end of the glass capillary. This end was placed in a H₂/O₂ flame and the glass was melted around the Au wire. A microscope was used to confirm the Au wire was completely sealed by the melted glass and that no bubbles had formed around it. After the Au had been sealed in the glass, the W wire was secured to the glass capillary with an epoxy (Loctite, Henkel, Dusseldorf, Germany). The electrodes were again placed in an oven at 150° C. for 5 min to harden the epoxy. The melted capillary end containing the sealed Au wire was polished flat with successively finer grit sandpaper (180-, 400-, 800-, and 1200-grit Carbimet, Buehler, Lake Bluff, Ill.) and lastly with aluminum oxide powder (300 nm, Alfa Aesar, Ward Hill, Mass.) on a wetted felt pad. A Gamry Instruments Femtostat and Gamry Framework Version 4.10 software (Warminster, Pa.) was used for impedance measurements with the disk electrodes. The impedance was measured before surface modification in 0.1M NaCl. The electrode was allowed to adsorb overnight in a 0.2 wt % solution of a 3 mol % tyrosineamide copolymer solution in 0.1 M NaCl. A 0.6 V potential was applied for 5 min to the electrode in the polymer solution using chronocoulometry, after which the impedance was re-measured in 0.1 M NaCl.

Surface Plasmon Resonance. SPR was measured with the Spreeta SPR3 integrated three channel sensor module Model# TSPR1K23 (Nomadics, Stillwater, Okla.) using v. 20.97 of the Spreeta5 Multiple Channel Spreeta Program. The sensors were washed in 0.1M NaOH with 1% Triton X-100, rinsed with deionized water, and dried with flowing nitrogen gas prior to use according to the manufacturers instructions. They were calibrated in water at a refractive index of 1.3330. To apply a voltage to the SPR sensor surface for copolymer electrodeposition experiments, a 0.1 mm 99.99% gold wire was attached to the edge of the sensor with tape. The gold surface of the sensor served as the working electrode in the three-electrode configuration diagrammed in FIG. 2.

To monitor the adsorption of copolymers onto gold, calibrated Spreeta sensor modules were placed in 0.2 wt % solutions of the 0-3 mol % tyrosineamide copolymers (FIG. 2). After approximately 2 hrs of copolymer adsorption, the sensor was either returned to water or a 0.6 V potential was applied for 5 min before returning the sensor to water. After polymer deposition by either passive adsorption or electrochemical deposition, the sensors were washed in 0.1 M NaOH with 1% Triton X-100 for 10 minutes.

Protein binding. A recombinant His₆-peptide tagged green fluorescent protein (GFP-H₆) was used as a model protein for studies of protein adsorption to the gold SPR sensor surface. GFP-H₆ binding to copolymer modified and unmodified gold surfaces was monitored by SPR. Non-specific adsorption of GFP-H₆ to unmodified and copolymer modified sensors was monitored by transferring SPR sensors equilibrated in phosphate buffered saline (PBS) pH 7.4 to solutions of 0.05 mg/ml GFP-H₆ in PBS. After 20 minutes the sensors were washed three times with PBS and then monitored until a steady SPR baseline (in refractive index, RI, units) was achieved. Cotyrosineamide modified sensors were prepared by electrodepositing a 3 mol % tyrosineamide copolymer onto the surface as described above.

Specific immobilization of GFP-H₆ was tested with a sensor modified by electrodeposition of polyacrylamide-co-tyrosineamide-co-MABNTA. The RI was monitored as the sensor was first placed into a 0.05 mg/ml solution of GFP-H₆ in PBS (pH 7.4) to test for non-specific absorption. The sensor was washed and equilibrated with PBS and then metallated by incubation in a solution of 10 mM nickel acetate. After washing away excess nickel acetate with PBS, the sensor was again placed in the GFP-H₆ solution. To further demonstrate that the GFP-H₆ binding was specifically through His₆-Ni(II) complexes the sensor was washed twice with 100 mM EDTA in PBS to sequester Ni(II) and reequilibrated in PBS.

Specific immobilization of GFP-H₆ was verified using fluorescent microscopy. Planar gold electrodes (1 cm square) were dipped halfway into a solution of polyacrylamide-co-tyrosineamide-co-MABNTA and a 0.6 V potential was applied. After copolymer electrodeposition, the electrodes were washed with water several times. For specific immobilization the copolymer modified electrodes were soaked in 10 mM nickel acetate solution in water for 10 min followed by washing. Metallated and unmetallated electrodes were placed in a 0.05 mg/ml GFP-H₆ solution for 20 min and then washed three times with PBS. The electrodes were examined by fluorescence microscopy. The images were digitized and the average pixel intensity was determined using ImageJ image processing software. To confirm specificity of binding, the electrodes with Ni(II) bound GFP-H₆ were washed three times with 100 mM EDTA and reexamined by fluorescence microscopy.

### II. Results

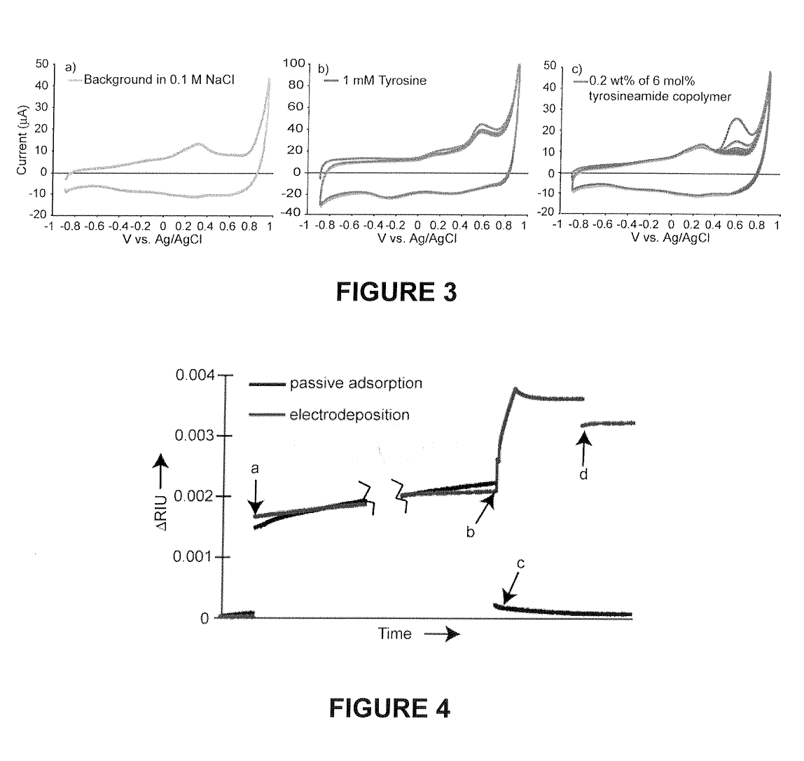

Cyclic voltammetry. Cyclic voltammetry (CV) with gold electrodes in solutions of 0.2 wt % copolymer containing 6.0 mol % tyrosineamide sidechains resulted in irreversible oxidation peaks at ˜0.6 V vs. Ag/AgCl (FIG. 3c). The peaks occurred at a similar voltage as oxidation peaks observed during cyclic voltammetry of free tyrosine (FIG. 3b). In contrast to free tyrosine, the magnitude of the oxidation peak of the tyrosineamide copolymer was diminished in each subsequent scan cycle. Contact angles measured before and after CV of the tyrosineamide copolymer solution demonstrated that the initially hydrophobic gold surface became hydrophilic (data not shown). The impedance of a 0.1 mm gold disc electrode measured at 1 kHz before and after CV in the tyrosineamide copolymer solution increased from 112 kOhm to 177 kOhm. Together, these observations suggested that a hydrophilic surface layer of the tyrosineamide copolymer was electrochemically deposited on the electrodes that passivated the gold surface to further tyrosineamide oxidation.

Adsorption and electrodeposition of tyrosineamide copolymers. When a Spreeta SPR sensor module was placed in a 0.2 wt % solution of a 3 mol % tyrosineamide copolymer (FIG. 4, arrow a) the refractive index increased as the copolymer passively adsorbed onto the sensor surface. After 2 hrs of adsorption (arrow b) the refractive index returned to near baseline when the sensor was washed with 0.1 M NaOH and 1% Triton X-100 (blue line, arrow c). When a 0.6V potential was applied for 5 min (pink line, arrow b) the refractive index increased dramatically. Washing with 0.1M NaOH and 1% Triton X-100 (arrow d) removed a comparatively small amount of the copolymer in contrast to the passively adsorbed copolymer. The stability of the electrodeposited tyrosineamide copolymer layer under harsh washing conditions suggested a robust attachment to the gold sensor surface.

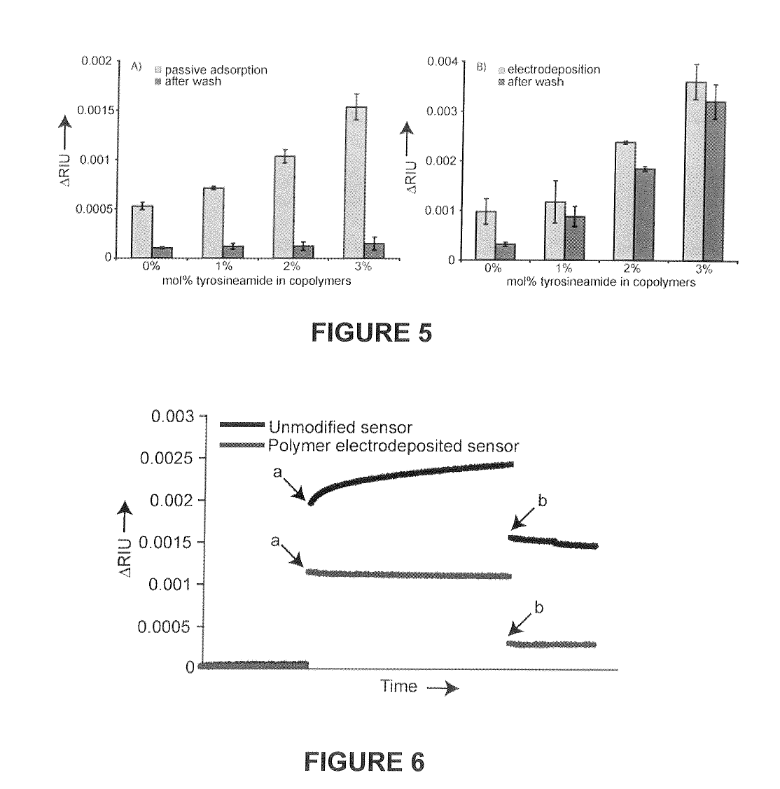

Additional SPR experiments demonstrated a correlation between the amount of tyrosineamide in the copolymer with the mass of adsorbed copolymer over the range of 0 to 3 mol % tyrosineamide demonstrating that the tyrosineamide sidechains are responsible for adsorption (FIG. 5A). In each case the passively adsorbed copolymer could be almost entirely removed by washing the sensor surface with 0.1 M NaOH and 1% Triton X-100 Likewise, an increasing mol % of tyrosineamide sidechains led to increasing amounts of electrodeposited copolymer resistant to harsh washing conditions (FIG. 5B).

Non-specific protein binding. A refractive index baseline was established by equilibrating a clean, unmodified SPR sensor in PBS (pH 7.4) before transfer to a 0.05 mg/ml solution of GFP-H₆ in PBS (FIG. 6, blue line, arrow a). After 2 hr the sensor was returned to PBS (arrow b). The refractive index remained substantially above the baseline value indicating that GFP-H₆ had bound non-specifically to the hydrophobic gold surface. In contrast, the initial refractive index change was much lower when a sensor pre-coated with a 3 mol % tyrosineamide copolymer layer by electrodeposition was incubated in 0.05 mg/ml GFP-H₆ (FIG. 6, pink line, arrow a). When re-equilibrated in PBS buffer (arrow b) the final change in refractive index from the baseline was 0.0002, about ⅛^{th} the final refractive index change of the unmodified sensor.

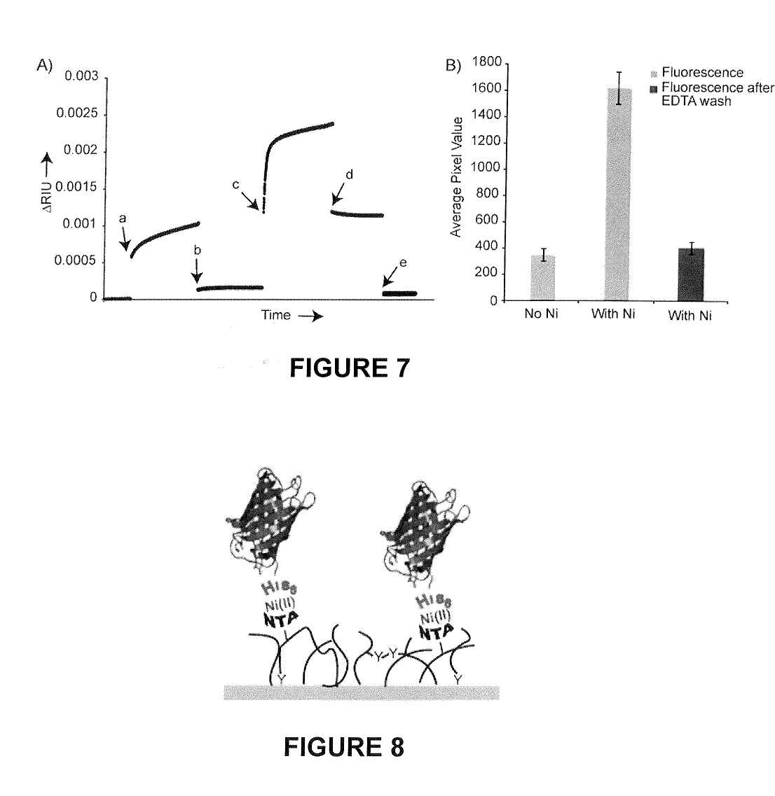

Specific protein binding. Sensors were modified by electrodeposition of acrylamide copolymers containing 3 mol % tyrosineamide and 3 mol % (2-methacrylamidobutyl)nitrilotriacetic acid (MABNTA) sidechains (FIG. 8). When incubated with 0.05 mg/ml GFP-H₆ (FIG. 7A, arrow a) the refractive index increased initially, but returned to near baseline when re-equilibrated with PBS buffer (arrow b). Following metallation of the NTA-containing sensor surface with Ni(II) the refractive index increased dramatically when incubated with 0.05 mg/ml GFP-H₆ (arrow c). When washed and re-equilibrated with PBS buffer the refractive index remained elevated (arrow d). The difference in RIU between arrow d and arrow b represents specifically bound GFP-H₆. To demonstrate that GFP-H₆ binding was specifically through metal coordination bonds between [NTA-Ni(II)] and the His₆ tag, the sensor surface was washed with 0.1 M EDTA to sequester the Ni(II) and disrupt metal coordination bonds. The refractive index returned to near baseline as expected (arrow e). The specific binding was confirmed by quantifying the fluorescence of GFP-H₆ immobilized on a gold sputtered electrode coated with cotyrosineamide-co-MABNTA acrylamide (FIG. 7B). After metallating the electrode surface with Ni(II), 4-5 fold more GFP was bound than without Ni(II). Furthermore, the bound GFP was nearly entirely removed by chelating the Ni(II) with EDTA.

### Kinetic Measurements Using SPRi

SPRi was used to monitor the adsorption of polymers and proteins to gold films in order to verify the results from other experimental methods, including impedance measurements and SPR measurements using a commercially available system from Spreeta. Neither the impedance nor the Spreeta SPR measurements could monitor the adsorption process in real time, and real-time analysis is necessary in order to more accurately determine the kinetic binding constants and to determine the amount of time needed for saturation with a single experiment. The experiment involved the passivation of gold-patterned electrodes with a polymer containing a nickel-binding nitrilotriacetic acid (NTA) side chain to a gold film in a region-specific manner. The NTA side chain then allowed for subsequent His₆-tagged protein binding using nickel as a chelator between the polymer and the protein. The passivation and protein binding processes were monitored in situ, and the kinetic constants for protein binding were determined from the SPRi data. Included in the research is the optimization of polymer removal from non-passivated electrode surfaces using harsh washing conditions. Ultimately, the procedure will be used as a unique way to pattern gold-covered substrates with specific proteins or other biomolecules.

Site-directed polymer and protein adsorption. A simple flow cell with a gasket separating the substrate from the flow cell was employed for these experiments. It is important to note that harsh washing conditions should remove any polymer not electropolymerized, but should not affect polymer which has been electropolymerized to the gold surface. Four control experiments were performed initially. The first control experiment simply tested the adsorption of protein to a single pair of gold electrodes which had been passivated ex situ. The purpose of the EDTA addition was to chelate the nickel, leading to desorption of the protein. The results of adsorption to both the passivated and non-passivated electrodes are shown. The various fluids (water, nickel, water, PBS, GFP, water, EDTA, water) were flowed across the gold surface. The results for the passivated electrode indicate that the protein was removed following the addition of EDTA (FIG. 11). For the non-passivated electrode, the results indicate that GFP had bound to the gold in a non-specific manner, and remained adsorbed to the gold even after the addition of EDTA.

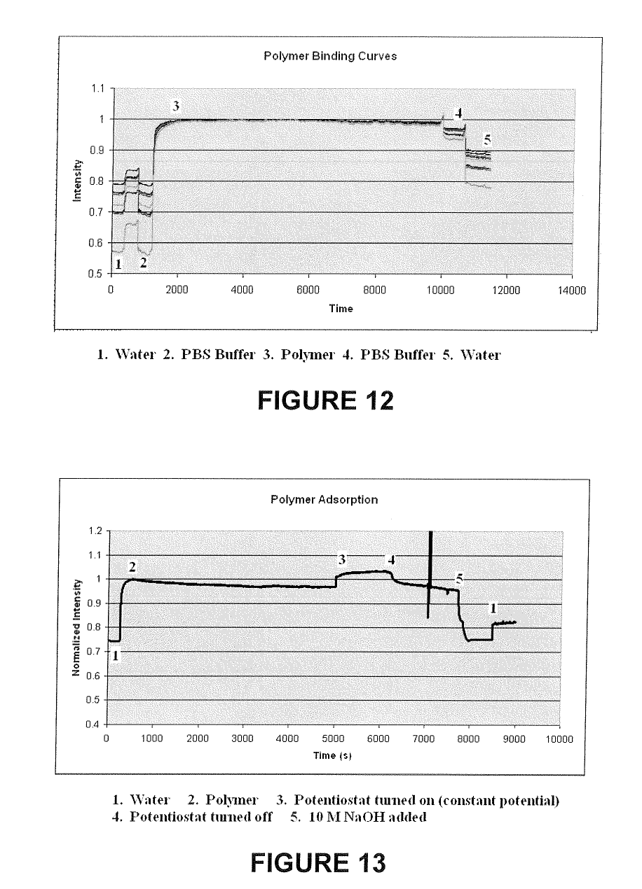

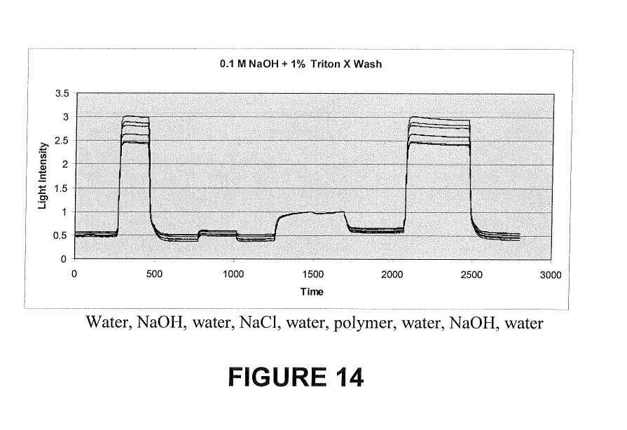

The second control experiment tested the adsorption of polymer to the gold film in the absence of harsh washing conditions. FIG. 12 shows the experiment. The results indicate that the polymer remained on the gold electrode if harsh washing conditions are not used. This indicated that optimization of washing conditions would be required to completely remove any polymer not electrochemically deposited on the electrode. In the third control experiment, the electropolymerization process was monitored in situ, and the results are shown in FIG. 13. For this experiment, a glass substrate with one pair of electrodes was used. Although data from this experiment implied that the electrode was passivated (i.e., polymer was electrochemically deposited on the electrode) another control experiment was necessary to ensure that polymer would be removed in the absence of electropolymerization. For this experiment, the polymer was adsorbed as previously, and then washed with 0.1 M NaOH and 1% Triton X without being subjected to the electrodeposition. The results of the experiment are shown in FIG. 14. The results indicate that a significant amount of polymer remained on the gold surface, even when subjected to harsh washing conditions. More experiments need to be done in order to optimize the washing conditions. The presence of polymer on the gold was verified with ellipsometry for some of the experiments (see below).

Site-directed polymer and protein adsorption. The results from the various experiments indicate that the electrodeposition process results in an increased amount of adsorbed polymer, but a change in washing conditions will be necessary in order to completely remove polymer from non-passivated electrodes. The following table contains the calculated values for intensity change and surface coverage determined from the SPR data, as well as thicknesses determined with ellipsometry:

| | Intensity Change | Surface Coverage | Ellipsometry |
|---|---|---|---|
| Experiment: | (ΔI, %): | (molecules/cm²): | Thickness (nm): |
| Passive adsorption, water wash | 2.1 | 8.4 × 10¹³ | 60.45 ± 3.25 |
| Passive adsorption, | 0.77 | 3.1 × 10¹³ | 5.2 ± 0.20 |
| 0.1 M NaOH, 1% Triton X | | | |
| Electropolymerization, | 3.3 | 1.7 × 10¹⁴ | — |
| 0.1 M NaOH, 1% Trition X | | | |
| Protein Adsorption After EDTA | 0 | 0 | — |
| Addition (Passivated): | | | |
| Protein Adsorption After EDTA | 16 | 1.06 × 10¹³ | — |
| Addition (Non-passivated)*: | | | |

*Surface coverage is dependent on the refractive indices and specific volume of the adsorbate. The intensity change for protein adsorption was higher than for the polymer, but surface coverage was still lower.

Throughout this application, various publications are referenced. The disclosures of these publications in their entireties are hereby incorporated by reference into this application in order to more fully describe the compounds, compositions and methods described herein.

Various modifications and variations can be made to the compounds, compositions and methods described herein. Other aspects of the compounds, compositions and methods described herein will be apparent from consideration of the specification and practice of the compounds, compositions and methods disclosed herein. It is intended that the specification and examples be considered as exemplary.

1. A method for applying a polymer to a metal substrate comprising electrochemically depositing a polymer on the surface of the metal substrate, wherein the polymer comprises at least one aromatic group, and wherein the aromatic group comprises one hydroxyl group.

2. The method of claim 1, wherein the aromatic group comprises a phenyl group.

3. The method of claim 1, wherein the polymer comprises at least one ortho- or meta-phenolic group.

4. The method of claim 1, wherein the polymer comprises at least one para-phenolic group.

5. The method of claim 1, wherein the aromatic groups further comprises at least one halogen group.

6. The method of claim 1, wherein the polymer comprises a polyester backbone, a polyalkylene backbone, a polyalkylene oxide backbone, or a polysaccharide backbone.

7. The method of claim 1, wherein the polymer comprises a polyacrylate comprising one or more pendant aromatic groups, wherein at least one of the aromatic group comprising one hydroxyl group.

8. The method of claim 1, wherein the polymer comprises at least one fragment comprising the formula I 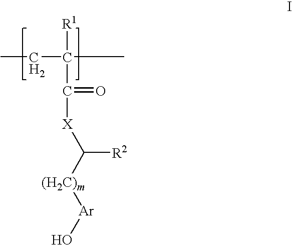
wherein R¹ is hydrogen or an alkyl group;
R² is hydrogen, C(O)NH₂, or C(O)OH;
X is O or NR³, wherein R³ is hydrogen or an alkyl group;
m is from 1 to 10;
Ar is an aromatic group,
or the pharmaceutically-acceptable salt or ester thereof.

9. The method of claim 8, wherein R¹ is hydrogen or methyl, X is NH, m is 1, R² is C(O)NH₂, Ar is a phenyl group, and the hydroxyl group is at the para position of the phenyl group.

10. The method of claim 1, wherein the polymer comprises the polymerization product between one or more ethylenically unsaturated monomers, wherein the aromatic group comprising the hydroxyl group is directly or indirectly attached to the polymer backbone.

11. The method of claim 10, wherein the ethylenically unsaturated monomer comprises an acrylate, a methacrylate, a vinyl compound, an allyl compound, or any combination thereof.

12. The method of claim 10, wherein the aromatic group comprising the hydroxyl group is indirectly attached to the polymer backbone by a linker.

13. The method of claim 12, wherein the linker comprises a methylene group, an ether group, or a polyamine group.

14. The method of claim 1, wherein the polymer comprises the copolymerization product between (1) a compound comprising the formula II 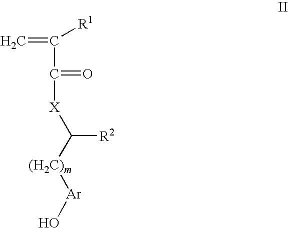
wherein R¹ is hydrogen or an alkyl group;
R² is hydrogen or C(O)NH₂ or C(O)OH;
X is O or NR³, wherein R³ is hydrogen or an alkyl group;
m is from 1 to 10;
Ar is an aromatic group,
or the pharmaceutically-acceptable salt or ester thereof; and
(2) acrylamide, acrylic acid or derivative thereof, methacrylic acid or derivative thereof, and any combination thereof.

15. The method of claim 14, wherein R¹ is methyl, X is NH, R² is C(O)NH, m is 1, Ar is a phenyl group, and the OH group is at the para-position of the phenyl group.

16. The method of claim 1, wherein the polymer comprises a biomolecule having at least one aromatic group, wherein the aromatic groups comprises at least one hydroxyl group.

17. The method of claim 16, wherein the biomolecule comprises a protein, peptide, oligonucleotide, or a small molecule.

18. The method of claim 16, wherein the biomolecule comprises a protein or peptide comprising one or more tyrosine residues.

19. The method of claim 1, wherein the polymer comprises one or more ligands covalently attached to the polymer.

20. The method of claim 19, wherein the ligand comprises a biomarker.

21. The method of claim 20, wherein bioactive agent comprises a peptide, a protein, an oligonucleotide, or a small molecule.

22. The method of claim 1, wherein the polymer comprises one or more amino acids covalently attached to the polymer.

23. The method of claim 22, wherein the amino acid comprises tyrosine or tyrosineamide.

24. The method of claim 1, wherein the metal comprises platinum, palladium, titanium, or iridium.

25. The method of claim 1, wherein the metal comprises gold.

26. The method of claim 1, wherein the electrochemical deposition step is performed at a potential sufficient to oxidize at least one aromatic group.

27. A polymer-modified metal substrate produced by the method of claim 1.

28. A medical device comprising a metal substrate having at least one surface and a polymer electrochemically deposited on the surface of the metal substrate, wherein the polymer comprises at least one aromatic group, and the aromatic group comprises one hydroxyl group.

29. A biosensor comprising a metal substrate having at least one surface and a polymer electrochemically deposited on the surface of the metal substrate, wherein the polymer comprises at least one aromatic group, and the aromatic group comprises one hydroxyl group.

30. The biosensor of claim 29, wherein the biosensor comprises one or more electrodes, wherein each electrode comprises a metal surface with the polymer electrochemically deposited on the surface.

31. The biosensor of claim 30, wherein each polymer is the same or different.

32. The biosensor of claim 29, wherein the biosensor comprises a surface plasmon resonance detector.

33. The biosensor of claim 29, wherein each electrodeposited polymer comprises a specific ligand that specifically binds a biomolecule.

34. The biosensor of claim 33, wherein the ligand is the same or different for each electrode.

35. A method for producing a biosensor comprising two or more electrodes, wherein the electrodes comprise a metal substrate, the method comprising:
(a) applying a first polymer on the surface of a first electrode, wherein the first polymer comprises at least one aromatic group, and wherein the aromatic group comprises one hydroxyl group;
(b) applying a first potential to the first electrode to electrochemically deposit the first polymer on the first electrode;
(c) washing the first and second electrode to remove undeposited first polymer;
(d) applying a second polymer on the surface of a second electrode, wherein the second polymer comprises at least one aromatic group, and wherein the aromatic group comprises one hydroxyl group;
(e) applying a second potential to the second electrode to electrochemically deposit the second polymer on the second electrode; and (f) washing the first and second electrode to remove undeposited second polymer.

36. A method for monitoring the electrodeposition of a polymer on the surface of a metal substrate, the method comprising (a) electrochemically depositing a polymer on the surface of the metal substrate, wherein the polymer comprises at least one aromatic group, and wherein the aromatic group comprises one hydroxyl group, and (b) using surface plasmon resonance spectroscopy to monitor the electrodeposition of the polymer during step (a).

37. A method for monitoring the absorption of a biomolecule by a polymer electrochemically deposited on a metal substrate, the method comprising (a) contacting the electrochemically deposited polymer with the biomolecule, and (b) using surface plasmon resonance spectroscopy to monitor the binding of the biomolecule to the electrochemically deposited polymer.

38. The method of claim 36, wherein the surface plasmon resonance spectroscopy comprises surface plasmon resonance microscopy or surface plasmon resonance imaging.
